(19) United States
(12) Patent Application Publication
(10) Pub. No.: US 2009/0041826 A1
(43) Pub. Date: Feb. 12, 2009

(54) Tissue Implant and Process for Its Production

(75) Inventors: Michael Jagodzinski, Hannover (DE); Carl Haasper, Hannover (DE); Christian Krettek, Hannover (DE)

Correspondence Address: GREER, BURNS & CRAIN, 300 S WACKER DR, 25TH FLOOR, CHICAGO, IL, 60606, US

(73) Assignee: MEDIZINISCHE HOCHSCHULE HANNOVER, Hannover (DE)

(21) Appl. No.: 12/083,648

(22) PCT Filed: Oct. 13, 2006
(86) PCT No.: PCT/EP2006/067406

§ 371 Date: Apr. 15, 2008

(30) Foreign Application Priority Data
Oct. 17, 2005 (DE) 10 2005 049905.8

(51) Int. Cl. A61L 27/38; C12M 3/00

(52) U.S. Cl. 424/426; 424/93.7; 435/297.1

(57) ABSTRACT

The present invention relates to a process for producing an implant for medical purposes, to the implant obtainable by the process, and to the bioreactor used for the production of the implant. The implants of the invention can be used as implantable prostheses having essentially the functional characteristics of the following tissues: ligaments and tendons, bone and chondrogenic tissues, e.g. intervertebral discs, and combinations of bone tissue with cartilage tissue, e.g. sections of joints having the cartilaginous surface of a joint surface hacked by bone tissue. According to the invention, a porous matrix material is incubated under cell culture conditions with cells while the matrix is exposed to positive pressure on one side and negative pressure on another side for effective colonization of the matrix.

The present invention relates to a process for producing an implant for medical purposes, to the implant obtainable by the process, and to the bioreactor used for the production of the implant.

Medical uses for the implant according to the invention are prosthetic purposes, e.g. replacing tissue defects. The implants of the invention can be used as implantable prostheses having essentially the functional characteristics of the following tissues: ligaments and tendons, bone and chondrogenic tissues, e.g. intervertebral discs, and combinations of bone tissue with cartilage tissue, e.g. sections of joints having the cartilaginous surface of a joint surface backed by bone tissue.

### STATE OF THE ART

WO 95/018810 focuses on the production of ligament implants by cultivating a bioresorbable matrix material with a fibroblast cell suspension. For seeding the matrix material with cells, the application of suction or centrifugal force is proposed to seed the cells into the porous matrix. In order to obtain the desired cell differentiation and cellular structure within the seeded matrix material, incubation under cell culture conditions is performed in an apparatus suitable for straining the matrix along a single longitudinal axis, namely applying a pulling force to the seeded matrix material.

DE 103 49 484 A1 discloses a bioreactor for cultivating three-dimensional structurally stable implant devices. The bioreactor allows the perfusion of the porous matrix seeded with cells obtained from the future recipient of the implant while periodically applying pressure onto the matrix by the movement of two opposed moulds. For the generation of mechanical pressure onto the matrix, one of the moulds is actuated electro-mechanically by a magnetic field generated outside the bioreactor while the mould comprises a co-operating magnet.

DE 199 62 456 A1 describes a bioreactor for pressing an aqueous matrix suspension, e.g. of collagen, comprising chondrocytes between opposed moulds. The moulds may be replaced by porous ceramic plates, one surface of which is arranged adjacent the pressed matrix, the opposed surface being exposed to circulating cell culture medium.

DE 102 41 817 A1 describes an implant produced by pressing an admixture of collagen fibres with cartilage cells. For pressing the aqueous suspension of collagen, a pressing mould arranged in a cylinder is used.

DE 198 08 055 A1 describes the production of tissue pellets by using centrifugal force for depositing cells onto a carrier material. Subsequently, a bottom section of the centrifugation vessel is introduced into a flow chamber to continuously provide fresh medium to the cells. For exerting mechanical compression, moulds are arranged adjacent the pellets.

DE 100 62 626 A1 describes a process for the production of a three-dimensional matrix material of collagen, embedded in which cells are cultivated in vitro.

DE 100 62 623 A1 describes the production of a dermis equivalent by cultivating fibroblasts onto three-dimensional aqueous collagen.

### OBJECTS OF THE INVENTION

In view of the known processes for the generation of implants having a predetermined three-dimensional structure, it is an object of the present invention to provide improved implants.

Further, it is an object of the present invention to provide an alternative process for the production of an implant having a predetermined three-dimensional shape comprising cells.

Additionally, it is an object of the present invention to provide an alternative bioreactor for use in the production of implants, the bioreactor preferably having a design that avoids the occurrence of infections during or after the period of cell culture. More preferably, the bioreactor is devoid of complex mechanical actuating systems while still exerting alternating pressure regimens onto the implant during its cultivation.

### GENERAL DESCRIPTION OF THE INVENTION

The present invention attains the above-mentioned objects by providing an implant containing cells in intimate conjunction with a porous matrix, e.g. an implant wherein the cells are essentially present within the volume of the porous matrix material, and by providing the process for producing the implant using the novel bio-reactor.

The novel bioreactor provides for an effective infiltration of the cells into the porous matrix material, during which infiltration the cells can be present in admixture with a solution or dispersion of a viscous component, e.g. with an aqueous composition of collagen. Further, the novel bioreactor allows to perform a cultivation process under constant or periodically changing mechanical strain in the form of positive pressure exerted onto the porous matrix material.

In a further embodiment, the present invention relates to implants having an essentially non-porous matrix material, which implant is cultivated in the bioreactor according to the invention under constant and/or periodically altering strain exerted in the form of stretching of the matrix material.

In general, the matrix contained in the implants according to the invention provides for an initial mechanical stability and serves as a scaffolding for cells to adhere to under in vitro cultivation conditions.

In a first embodiment, the matrix is an essentially non-elastic, structurally stable body, e.g. a porous matrix selected from the group comprising acellularised tissue, acellularised bone, acellularised spongiosa, and acellularised veins, venules, tendons or ligaments, preferably of a thickness of 1 to 2 mm. Acellularized material can be allogenic or xenogenic, e.g. obtained from animals. Alternatively, synthetic bioresorbable materials can be used for forming the porous matrix material, e.g. calcium-hydroxyl apatite, calcium-deficient hydroxyl apatite, β-tri-calcium phosphate, bioresorbable polymers or ceramics.

In the second embodiment, the matrix is an elastic body, which is not necessarily porous, but which has a resilience or recovery, e.g. a filament selected from the group of natural and synthetic tendons, ligaments and sutures. The matrix having resilience also serves as a support for the cells to adhere to, also predetermining the shape of the final implant.

For the production of the implant being colonized with cells, which are preferably autologous to the future implant recipient, cell types are used that occur in the natural body section to be supplemented or replaced by the implant. Accordingly, the final implant preferably contains cell types originating from the future implant recipient, i.e. autologous cells, for forming a supplement or replacement of the body section which is defective. Optionally, the cells obtained from the future implant recipient, e.g. from a tissue sample obtainable by biopsy, are preselected and/or enriched prior to adhering to and colonizing the matrix material.

Examples of cells suitable specific implants are comprised in the following table, which is a non-limiting enumeration of implants.

TABLE
Combinations of matrix material and cells for implants

| implant/defect to | matrix | |
|---|---|---|
| be supplemented | material | autologous cells |
| bone | spongiosa | mesenchymal stem cells, |
| | | osteoblasts, optionally |
| | | in combination with |
| | | hematopoietic stem cells |
| | | and/or osteoclasts, |
| | | fibroblasts |
| cartilage tissue | collagen | chondrocytes, |
| | | fibrochondrocytes, |
| | | mesenchymal stem cells |
| composite implant | spongiosa | osteoblasts, followed by |
| comprising a layer | | chondrocytes and/or |
| of cartilage on bone, | | differentiated mesenchymal |
| e.g. for surfaces of | | stem cells in |
| joints | | admixture with collagen |
| tendon/ligament | acellularized | fibroblasts, mesenchymal |
| | venule, | stem cells |
| | acellularized | |
| | tendon or | |
| | acellularized | |
| | ligament | |
| tendon/ligament | suture, | fibroblasts, mesenchymal |
| | bioresorbable | stem cells |
| | polymer | |

The process for producing the implant uses a bioreactor having a first end section and an opposed second end section, e.g. formed by opposed ends of a tubular vessel, preferably having a circular cross-section. Between the first and second end sections, a fluid pervious carrier is arranged within the bioreactor, covering a portion of or essentially the complete the cross-section of the bioreactor's inner volume. The carrier is permeable to fluids, e.g. cell culture medium, and creates a flow resistance against its permeation of the fluid. In order to avoid flow of fluid around the carrier, the carrier is preferably provided with a circumferential sealing contacting the inner surface of the bioreactor.

The matrix material is arranged on a first side of the carrier, which first side is oriented towards the first end section of the bioreactor. For the introduction of cell culture medium into the bioreactor and for applying strain in the form of positive pressure onto the carrier, the first end section of the bioreactor is provided with an inlet. The second end section is provided with an outlet for receiving and withdrawing cell culture medium after passing through the carrier and through and/or around the matrix material arranged on the carrier.

According to the invention, the introduction of medium by the inlet at the first end section to exert positive pressure onto the carrier is combined with the withdrawal of medium from the outlet at the second end section for exerting negative pressure onto the second surface of the carrier, facing the second end section. This combination of positive pressure onto the first side of the carrier and negative pressure onto the second side of the carrier is preferably generated by at least partially circulating the cell culture medium exiting at the second end section back towards the first end section, using a fluid connection and a pumping means, e.g. tubing and a pump arranged between the second end section and the first end section. Preferably, the circulating pipe or tubing connecting the second end section with the first end section is formed by a closed line, and the pump is exerting its pumping action onto the fluid from outside the tubing, e.g. the pump is a flexible-tube pump. Optionally, the circulating line may be provided with or attached to a reservoir vessel for holding and/or conditioning, e.g. gassing/degassing the cell culture medium, and connected with a reservoir containing fresh cell culture medium and/or an outlet for withdrawing a portion of the circulating cell culture medium.

One of the advantages of the bioreactor and process performed with the bioreactor according to the invention is the simple design of the bioreactor, which is devoid of specific mechanical actuating means for exerting strain onto the matrix material, e.g. free from mechanical actuators. In contrast to known bioreactors, the bioreactor according to the invention is able to generate the strain onto the matrix material by applying positive pressure onto the carrier, which positive pressure is generated by the inflow of the cell culture medium.

For implants having a structurally stable matrix material and which are cultivated under constant or periodically occurring strain in the form of positive pressure, inflow of medium through the first end section of the bioreactor provides sufficient hydraulic pressure against the carrier. For matrix materials that are desirably cultivated under conditions in which the strain exerted is a pulling force, e.g. in the case of generating ligament or tendon implants, a first end of the matrix material is fixed to the region of the bioreactor adjacent its first end section, and the second end of the matrix material is fixed to the carrier. Again, the inflow of cell culture medium through the first end section of the bioreactor generates pressure onto the carrier, which pressure is translated in this embodiment into a strain pulling the matrix material, i.e. stretching the matrix material between its point of fixation at or near the first end section of the bioreactor and its point of fixation at the carrier.

In addition to the strain exerted during production of the implant, it is preferred to apply shear stress onto the matrix during cell culture in order to closer mimic the natural conditions and for obtaining more resistant and more stable implants. Shear stress can be generated and applied to the matrix by rotating the carrier in relation to the first mould by a fraction of a full circle, e.g. for an angle of 1 to 20° while contacting the matrix material. The relative rotation of the carrier and the first mould can be effected by guiding both the carrier and the first mould on a guide which along the length of the bioreactor, i.e. along the pathway of the relative movement of the carrier and the first mould in respect to each other, creates a rotational movement of at least one of carrier and first mould. For creating the rotational movement, the guide can have a spiral conformation extending along the axis between the first end section and the second end section of the bioreactor, or the guide can be arranged in an angle to this axis. As a consequence, a relative rotation between carrier and first mould is generated when the distance between carrier and first mould is changed, e.g. when the first mould is pressed against the matrix material arranged on the first side of the carrier. In the second embodiment, movement of the carrier along the axis between first end section and second end section generates a rotation of the point of fixation at the carrier with respect to the point of fixation at or near the first end section of the bioreactor and, hence, a twist occurs in the matrix arranged between these two points of fixation.

For the first embodiment, it is preferred that the first surface of the carrier has a surface conformation adapted to receive the adjacent surface conformation of the matrix material, preferably by positive fit. In addition, the first surface of the carrier can be sealed to be liquid tight in the region which is not covered by the adjacent surface of the porous matrix material in order to avoid the bypass of cell culture medium around the matrix material and through the carrier.

In a further improvement of the first embodiment, the bioreactor contains a first mould essentially extending across the cross-section of the bioreactor, having a first surface side oriented towards the first end section and a second surface side oriented towards the porous matrix material and towards the carrier, respectively. The first mould serves to improve the impregnation of the porous matrix material with viscous medium components, which is e.g. a solution or suspension of collagen. The second surface side of the first mould is provided with a conformation for receiving the conformation of the matrix material on a portion of the surface of the matrix material that is oriented towards the first end section. Preferably, the second surface side of the first mould is provided with the conformation having a positive fit with at least a surface section of the matrix material. The first mould is only actuated by the pressure exerted by the inflow of fluid through the first end section, without any requirement for a mechanical or electromechanical actuating means. The first mould is pervious to the fluid stream of medium to allow the flow of medium through its volume. However, the first mould has a porosity that generates a flow resistance sufficient for moving the first mould against the carrier and the porous matrix material, respectively, with a pressure. For registration of the first mould with the carrier such that their opposed surface conformations are properly oriented for positioning adjacent the matrix material arranged between them, a guide, e.g. a guide bead or guide notch or guide pin can be arranged along the length of the bioreactor to guide the carrier and the first mould. Accordingly, the carrier and the first mould can be provided with a respective guide receiving section, e.g. a notch, a bead or a boring for co-operating.

Apart from its construction allowing to generate the strain onto the matrix material to be colonized with cells during the cultivation without the need for mechanical or electro-mechanical actuating means, except for at least one pumping means, the bioreactor and the cultivation process performed with it have the advantage of producing an implant which is more thoroughly infiltrated and/or colonized with cells as compared to implants received in prior art reactors and processes.

The thorough infiltration and colonization of the porous matrix material in the bioreactor according to the invention is believed to be caused by the combination of positive pressure applied onto the porous matrix material arranged on the first side of the carrier in combination with the negative pressure, e.g. suction, applied from the second surface side of the carrier.

In addition, the bioreactor may be provided with one or more openings arranged in its perimeter for forming intermediate outlet openings. One or more intermediate outlet openings can be arranged at one or more distances between the first end section and the second end section. Preferably, the intermediate outlet openings are connected via fluid-tight lines to lines exiting from the second end section, preferably joining the line connected to the second end section before the low pressure side of a pumping means.

The intermediate outlet openings serve to at least partially automate the alternating, e.g. periodic generation of strain onto the carrier because movement of the carrier along the length of the bioreactor in the direction from the first end section towards the second end section will lead to passing of the carrier past the intermediate exit opening. As the pressure exerted onto the carrier is more easily released by the fluid exiting through the intermediate outlet opening than by passing through the porous matrix material and/or through the carrier, the pressure onto the carrier and/or onto the matrix material drops when the carrier has passed the intermediate outlet opening. The resilience of the matrix arranged between the first end section and the carrier after release of the pressure between the first end section and the carrier by the intermediate outlet opening forces the carrier into the direction of the first end section, positioning the carrier at a distance from the intermediate outlet opening towards the first end section. As a result, no fluid can exit through the intermediate outlet opening without passing the matrix material and the carrier, which leads to a pressure build-up on the first side of the carrier. This alternating build-up of pressure on the first side of the carrier and the pressure release through the intermediate outlet opening as soon as the carrier is pushed past the intermediate outlet opening creates a periodic strain onto the matrix material.

For regulation of the positive and negative pressures acting on the first and second side of the carrier, respectively, it is preferred that a valve is arranged in the fluid transporting lines connected to the second end section and/or to the intermediate outlet opening, either separately or after their junction.

For the transport of cells to and/or into the matrix material, a cell suspension is introduced at the first end of the bioreactor, or at an additional inlet port arranged between the first end and the carrier.

For an effective colonization of the matrix material, it is at present preferred to introduce autologous cells directly after biopsy, i.e. without intermediate cell culture. For direct introduction of biopsied cells into the matrix material, the heparinized cell suspension obtained by bone marrow aspiration, e.g. a puncture biopsy of the illiac crest, or heparinized blood is used. In addition, non heparinized blood or bone marrow aspirates can be injected in a second step forming a second layer in terms of a blood clot on top of the porous scaffold.

The fluid stream entering the bioreactor at the first end presses the medium including the cells suspended in the medium against the matrix material, allowing the fluid stream only to pass through the matrix material and the carrier, wherein the permeability of first side surface of the carrier is preferably restricted to the surface area contacted by the matrix material, which contact preferably is by positive fit. The positive fit between the porous matrix material and the carrier in a simple embodiment can be the alignment of planar surfaces, present in both the first side of the carrier and the adjacent surface of the matrix material. For restriction of the permeability of the carrier to a surface section contacting the matrix material, a section of the surface of the carrier can be sealed, e.g. by arranging a sealant or a sealing thereat.

The combination of the positive pressure acting onto the matrix material and the first side of the carrier in combination with the negative pressure acting on the second side of the carrier and the, accordingly, onto the surface of the matrix material contacting the carrier, causes the cells suspended in the medium to be effectively introduced into the volume of the matrix material.

The suspension of cells for colonizing the matrix material can further include suspended or dissolved compounds increasing the viscosity of the cell culture medium. An example for a component increasing the viscosity of the medium is collagen, preferably in the form of an admixture of collagen dissolved or suspended in cell culture medium containing cells for colonizing the matrix material.

In contrast to state of art bioreactors and production processes, the production process according to the present invention generates implants having cells, optionally in admixture with a viscosity increasing component, e.g. collagen, dispersed throughout the porous matrix material, whereas in the state of art processes, an admixture of cells and collagen in medium only results in the forming of a collagenous layer on a surface of the porous matrix material.

Accordingly, when subsequently incubating the cells introduced into the porous matrix material under cell culture conditions according to the present invention, cells proliferate within the porous matrix, colonizing its interior and exterior.

In the alternative to generating an implant using colonization of the porous matrix material with a single cell type or a mixture of different cell types, implants can be generated by subsequently introducing two or more different single cell types or cell type admixtures. For example, when generating a bone implant, the porous matrix material preferably is acellularised spongiosa, which is first colonized by a first cell type, and subsequently colonized by a second cell type. As the first cell type, a cell suspension obtainable from a biopsy, e.g. from a puncture biopsy of the bone marrow of the future recipient of the implant, is separated into two aliquots, one of which is immediately used for preparing an admixture with cell culture medium and introduced into the bioreactor of the invention, containing the spongiosa arranged on the carrier in positive fit.

Introduction of cell suspension from a puncture biopsy leads to the introduction of the admixture of cell types into the volume of the porous matrix material, and subsequent cultivation results in colonization of the outer surface and inner volume of the matrix material. It has been observed that the predominant cell type colonizing the matrix material is osteoblasts. For colonization with the first type of cells, a cultivation time of one to three weeks can be used. A second aliquot of the puncture biopsy is preferably subjected to Ficoll-gradient centrifugation for enriching mesenchymal stem cells. Alternatively, expanded chondrocytes can be generated and used. The mesenchymal stem cells, which optionally can be differentiated, and/or chondrocytes, optionally in admixture with other cell types, are preferably cultivated for expansion of cells, preferably in culture dishes in a so-called two-dimensional culture. In the following expansion of cells, e.g. up to a state where confluency starts to occur, the cells are collected for introduction into the bioreactor and collected, e.g. by trypsination followed by centrifugation. Before introducing these cells into the bioreactor, an admixture with a collagen suspension is preferably prepared. Introduction of the cultivated the Ficoll-gradient enriched cells in admixture with collagen into the bioreactor results in the infiltration of the matrix material, that is colonized with the first type of cells, with the second type of cells in combination with collagen. Following further cultivation in the bioreactor, while again applying periodically alternating positive pressure onto the carrier and negative pressure to the second side of the carrier, an implant is generated which in addition to colonization of the interior of the matrix material with the first cell type comprises a region substantially colonized with the second cell type introduced into the bioreactor, forming adhering cells and tissue comprising the second cell type at least over a substantial section of the volume of the matrix material. As described here, the second cell type being mesenchymal stem cells and chondrocytes, whereas the first of cell type are osteoblasts, the implant provides a cartilaginous surface and region at least over a substantial fraction of its surface and inner volume. Hence, an implant can be generated that can replace a section of a joint, comprising the cartilaginous surface.

In order to provide the desired shape necessary for substituting or filling the tissue defect in the future recipient of the implant, it is preferred that the conformation of the porous matrix material is shaped by computer assisted machining methods, e.g. on the basis of three-dimensional data obtained from the site of the future implant in order to fully adapt the three-dimensional conformation of the implant to the site of substitution for filling the tissue defect such as to provide the natural function of the respective tissue section. Instead or in addition to using measurements of the defective tissue, the healthy opposite side (e.g. alternate joint) to the defect is analysed for determining the three-dimensional shape required for the implant.

The present invention is now described in greater detail with reference to the figures, wherein

FIG. 1 schematically shows a bioreactor according to the invention,

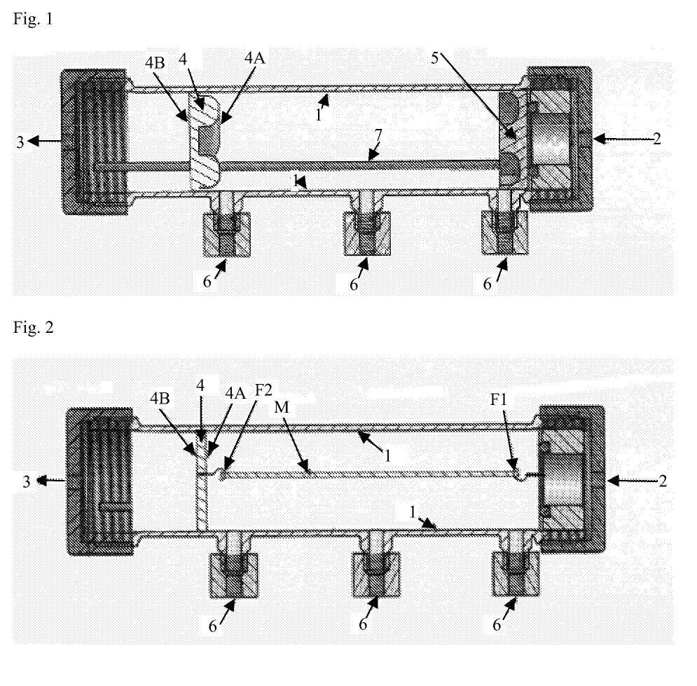

FIG. 2 schematically shows a bioreactor according to the invention for producing a resilient implant,

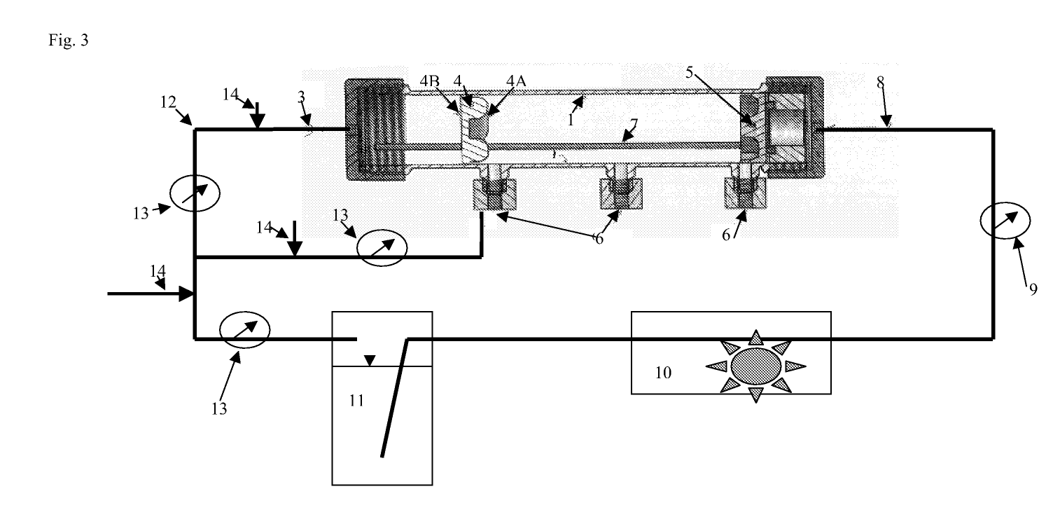

FIG. 3 schematically shows the arrangement feed and exit pipelines to the bioreactor,

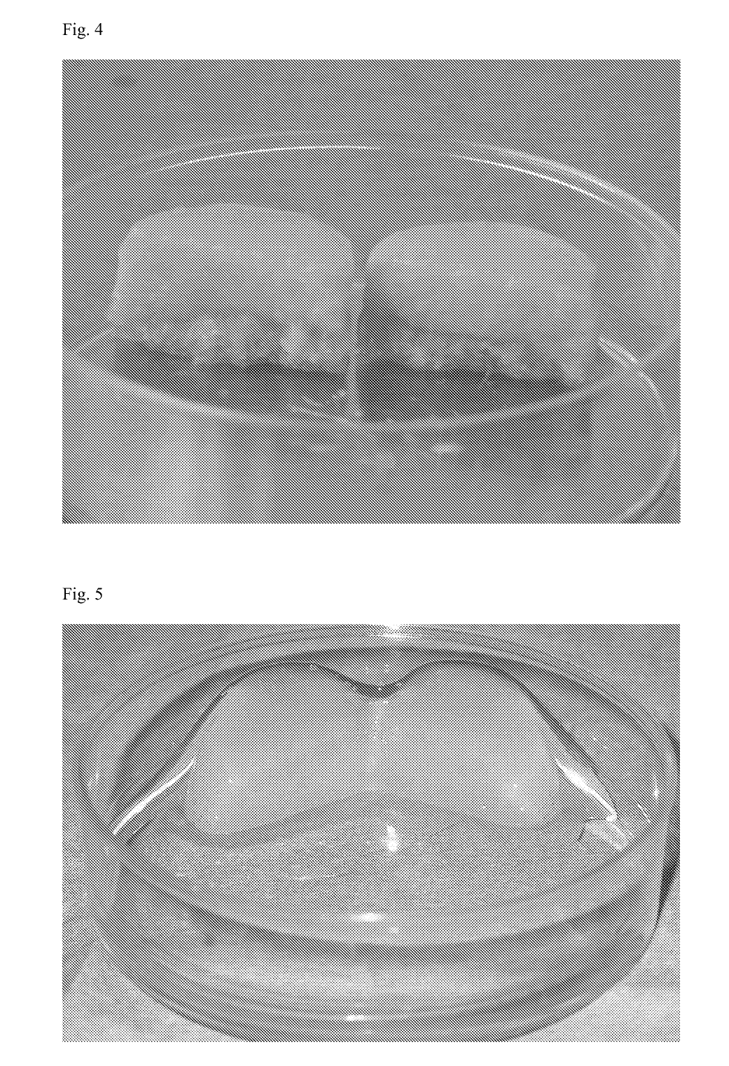

FIG. 4 shows the cross-section of an implant according to the invention,

FIG. 5 shows a comparative implant according to the state of art, and

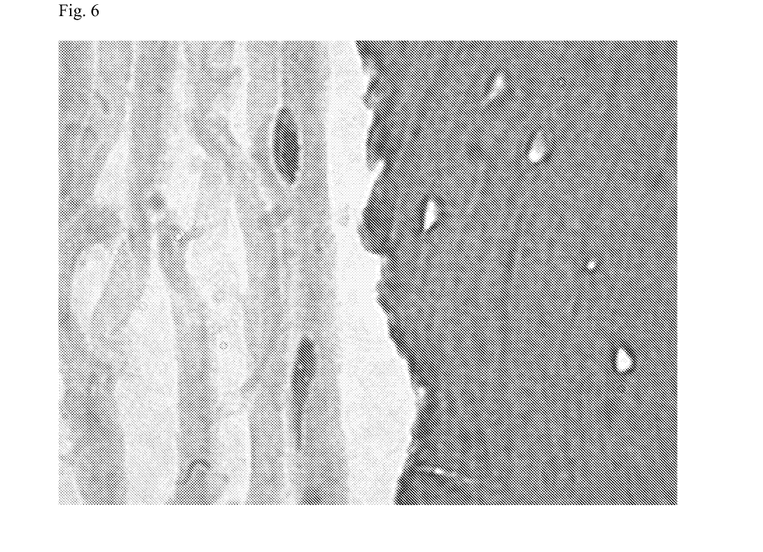

FIG. 6 shows a microscopic view of the boundary from spongiosa and cartilaginous tissue in an implant according to the invention.

### DETAILED DESCRIPTION

### Example 1

### Generation of a Bone Tissue Implant

From a puncture biopsy of bone marrow from the future recipient of the implant, autologous human adult bone marrow stem cells were obtained. After the biopsy, cells were immediately transferred into cell culture medium (Dulbecco's MEM, 10% fetal calf serum, which is preferably replaced by autologous human serum, and penicillin/streptomycin, amphotericin B, ascorbic acid).

Under sterile conditions, the cell suspension was layered over a Ficoll-gradient. Ficoll-gradient centrifugation (20 minutes, 4° C., 800×g) was followed by isolating the mononuclear white phase and pipetting into a fresh vessel. To the mononuclear phase, 30 mL PBS at 4° C. was added, followed by centrifugation for 10 minutes at 4° C. (480×g). The supernatant was removed and the cell pellet was resuspended in the same culture medium and cultivated in a culture dish in complete medium. Following five days incubation at 37° C., 5% CO₂ atmosphere, adherent cells were harvested by trypsination, washed under sterile conditions in PBS and finally resuspended in complete medium.

The cultivated cells were used for introduction into the bioreactor for colonization of the porous matrix material. In the alternative to cultivated bone marrow stem cells, the puncture biopsy was used as the cell fraction for introduction into the bioreactor or, as a further alternative, the mononuclear cell layer obtained by Ficoll-gradient centrifugation was used.

The bioreactor is schematically shown in FIG. 1. The bioreactor has an inner volume 1 having a circular cross-section, which inner volume is sealed off by the first end section 2 and the opposed second end section 3. The first end section 2 has at least one inlet port connected to a fluid line, e.g. tubing for introduction of fluids, whereas the second end section 3 has at least one exit port connected to a fluid line for withdrawal of fluid. The carrier 4 is shown to extend over the cross-section of the inner volume of the bioreactor. Intermediate openings 6 are arranged along the bio-reactor.

Carrier 4 is provided with a first surface 4A, conforming to the conformation of a porous matrix material (not shown) to be arranged adjacent carrier 4 in positive fit. When medium is introduced at first end section 2, surface 4A of the carrier 4 is exposed to pressure, which is generally in the range of up to 20 or 25 kPa, whereas the suction applied to second end section 3 generates a negative pressure acting on second surface 4B of carrier 4.

Preferably, the strain, e.g. the compression or pulling load acting onto the matrix material is a cyclic, sinusoidal force, e.g. a frequency of 0.1 to 2 or up to 5 Hz, e.g. a pressure onto the carrier in the range of 8 to 30 kPa, preferably 10 to 20 kPa.

A first mould 5, which is an option, is shown to cover the cross-section of the inner volume 1 of the bioreactor. Both the carrier 4 and first mould 5 are arranged moveably within inner volume 1 of the bioreactor such that the pressure exerted by fluid introduced at first end section 2 can move carrier 4 from the first end section 2 towards the second end section 3 and will move first mould 5 from the first end section 2 towards carrier 4. Depending on the positions of intermediate openings 6 and their resistance to fluid exiting, which flow resistance can be adjusted by valves attached to intermediate openings 6, the relative position of carrier 4 and first mould 5 with respect to each other and with respect to second end section 3 can be regulated. Further, the positioning of carrier 4 and first mould 5 with respect to each other can be predetermined by choosing their flow resistance, e.g. their thicknesses and porosities. In general, it is preferred that the flow resistance of first mould 5 is lower than the flow resistance of the carrier 4, causing the first mould 5 to be pressed against carrier 4 at lower pressures than necessary for moving carrier 4 close to second end section 3.

The carrier 4 and the first mould 5 are provided with recesses for receiving guide 7, depicted in the form of a guide rod. When shear stress is to be introduced into the matrix material, it is preferred that the guide rod has a helical conformation around the axis between the first end section and the second end section or that it is straight and inclined with respect to the axis between the first end section and the second end section.

In the alternative to using a carrier 4 adjacent the porous matrix membrane for creating a flow resistance against the flow of medium, and for separating the volume of the bioreactor into a positive pressure side and a negative pressure side with the matrix material being positioned at the interface of these pressure sides, the carrier 4 can be an integral portion of the matrix material. One example for this embodiment is a reactor design having a conformation which conforms to the perimeter of the matrix to form a sealing against cell culture medium flowing around the matrix material, which would be a short circuit that destroys separation of the positive pressure on one side of the matrix material and the negative pressure on the opposite side of the matrix material.

Further, independent of being realized as a separate part or forming an integral portion of the matrix material, the carrier 4 can be fixed to the walls of the bioreactor volume. In this embodiment, it is preferred that a moveable first mould is present in the bioreactor when exerting pressure onto a collagenous composition comprising cells towards the matrix material.

The porous matrix material was formed of an acellularised spongiosa, one surface conforming to the first surface 4A of carrier 4 in positive fit. When orienting the bioreactor such that the first surface 4A of carrier 4 is approximately oriented horizontally and upwards, no specific fixation of the porous matrix material towards carrier 4 is necessary.

The setup of the bioreactor for providing the circulation of cell culture medium is schematically shown in FIG. 3. Volume 1 of the bioreactor is provided with medium at its first end section 2 through line 8, which is provided with a metering device 9. In accordance with the preferred embodiment, the pumping means 10 is a pump exerting pressure without directly contacting the cell culture medium, here exemplified as a rotating head flexible-tube pump. Medium is withdrawn from a reservoir 11, which receives medium withdrawn from bioreactor volume 1 by line 12, which is connected to the second end section 3. Valves 14 are arranged in the withdrawal line 12 at a point downstream the junction of the line connected to second end section 3 with the line connected to intermediate opening 6 for controlling the negative pressure exerted onto second surface 4b of carrier 4.

For regulating the pressure generated by the pumping means 10, it is preferred to control pumping means 10 on the basis of signals obtained from metering devices 9 and 13, using a computer.

The cell culture medium was circulated from first end section 2 towards second end section 3 at the flow rate of 5 to 10 mL/min. The carrier 4 and first mould 5 were fabricated from porous glass, allowing the permeation of cell culture medium and of cells.

After cultivation for a period of 5 to 15 days, the acellularised spongiosa was colonized with cells that were initially introduced in suspension at first end 2. Colonization of spongiosa was found on the outer surfaces as well as in cavities of spongiosa within its inner volume.

### Example 2

### Production of a Joint Surface Implant Having a Cartilaginous Surface

Using the production process for of a bone implant according to Example 1, the implant was further provided with a cartilaginous surface. For the initial colonization of the spongiosa with bone marrow stem cells, an aliquot of the puncture biopsy was directly used for inoculating the bioreactor. Following the cultivation for a period of 10 to 15 days, including regular partial replacements of the medium outside the bioreactor, cartilaginous cells were introduced at first end section 2.

Cartilaginous cells were obtained by cell culture of the mononuclear cell fraction obtained by Ficoll-gradient centrifugation from an aliquot of the puncture biopsy, followed by cell culture for 5 to 12 days in the complete medium which was supplemented with insuline like growth factor 1 and transforming growth factor beta. After trypsination and washing, cartilaginous cells were carefully admixed with a suspension of collagen (2 to 5 mg/mL cell culture medium) to a final cell density of 10⁴ to 10⁶ cells/mL of collagen suspension in cell culture medium.

Following introduction of the cartilaginous cell suspension comprising collagen at the first end section 2 and after cultivation for an additional period of 10 to 15 days, the infiltration of cartilaginous cells in admixture with collagen into the volume of the spongiosa was effected by an initial phase of positive pressure onto the first surface of the carrier and negative pressure onto the second surface of the carrier for 12 to 36 h, preferably 24 h in order to obtain a reduction in volume by a factor of about 40 for the collagenous phase. This initial pressure/suction phase was followed by colonization of the spongiosa at its surface and within the inner volume of the spongiosa during cell culture under a cyclic sinusoidal pressure/suction regimen to the first and second surface of the carrier, respectively.

Using cylindrical spongiosa samples, infiltration of cartilaginous cells in admixture with collagen, was observed to start from the surface of the spongiosa oriented towards the first end 2 and covering a substantial portion of the inner volume of the spongiosa adjacent its outer surface.

A macroscopic cross-section of the spongiosa after cultivation with cartilaginous cells in admixture with collagen is shown in FIG. 4. The originally cylindrical sample was cut into two portions, demonstrating that cartilaginous cells in admixture with collagen colonized a depth of about two thirds of the inner volume of the porous matrix, starting from the upper surface that was oriented towards the first end section 2 of the bioreactor, whereas the bottom side of the implant sections shown in FIG. 4, now adjacent the bottom of the dish, were oriented adjacent the first side 4A of carrier 4.

During cultivation with the admixture of cartilaginous cells with collagen, first mould 5 was present in the bioreactor, having a shape conforming to the adjacent side of the spongiosa in positive fit.

In this example, the initial colonization of the matrix material with cells can be omitted to generate an implant having the cartilaginous tissue present on one surface and within the volume of the matrix material.

A detailed microscopic view across the edge region of the matrix material after cultivation with fibroblasts in admixture with collagen but without previous colonization with bone marrow stem cells is given in FIG. 6, demonstrating the close association of the cells with the collagen matrix and the porous matrix material.

### Comparative Example

### Colonization of Spongiosa with Cartilaginous Cells in Collagen Suspension According to the State of Art

Using the process described in Example 2, but without applying a negative pressure at second end section 3 of the bioreactor, an alternatively shaped spongiosa having a similar maximum thickness was incubated. Again, both carrier 4 and first mould 5 were provided with conformations such that the respective surfaces adjacent the spongiosa had positive fit with the spongiosa.

As a result, it was observed that the lack of negative pressure acting on second side 4B of carrier 4 resulted no infiltration and essentially no colonization of the cartilaginous cells in admixture with collagen into the volume of the spongiosa. This comparative example is shown in FIG. 5, demonstrating that the collagen matrix could not enter the volume of the spongiosa but forms a separate layer on top of the outer surface of the spongiosa only, which layer is not tightly connected to the matrix material. Accordingly, the layer of cartilaginous cells obtained according to the state of art was not suitable for replacing a section of a joint surface.

### Example 3

### Production of a Tendon Implant

For production of a tendon implant, a bioreactor according to FIG. 2 was used, wherein the bioreactor was provided with a fixation apparatus, e.g. a hook, adjacent first end section 2, and carrier 4 was provided with a fixation apparatus on its first surface 4A, both fixation apparatuses 4 receiving one end of the matrix.

As the matrix material M, a porous matrix was used, namely an acellularised venule or an acellularized xenogenic ligament, alternatively, a non-porous matrix, namely a fibre selected from the group comprising sutures was used. The matrix material was attached to the first fixation apparatus F1 and the second fixation apparatus F2.

For cultivation, the bioreactor was provided with a flow of 10 mL/min cell culture medium, to which initially cells were added for adhering to and colonizing the matrix. Suitable cell types were chosen from differentiated mesenchymal stem cells or fibroblasts, each of which were optionally previously expanded by two-dimensional cell culture, e.g. using cells obtained from a skin biopsy.

Presently, it is preferred to use heparinized full blood, as this contains stem cells, or bone marrow aspirate, e.g. obtained by illiac crest puncture biopsy directly, i.e. optionally without Ficoll-gradient centrifugation and especially without any cell cultivation previous to colonizing the matrix material.

Depending on the initial resilience and break resistance of the matrix material, the fluid stream was adjusted such that the pressure onto carrier 4 only generated a pulling force to the matrix material below its break resistance.

In general, the inflow of medium at the first end section 2 and withdrawal of medium at the second end section 3 was generated by a pump integrated into a circulating line. The application of positive and negative pressure on carrier 4 was periodical, namely by operating the pumping mechanism at intervals only, leaving intervals without fluid pressure applied. Within these intervals of rest, the matrix could at least in part return to its shape without strain applied, according to its resilience.

1. Implant comprising a porous matrix material and cells, obtainable by
cultivating the porous matrix material in a bioreactor having a carrier pervious to fluids, which carrier is covering the cross-section of the bioreactor inner volume and is arranged between a first end section and an opposed second end section of the bioreactor inner volume, the carrier having a flow resistance against the permeation of a fluid,
wherein the porous matrix material is arranged on a first surface of the carrier oriented towards the first end section,
introducing the cells under cell culture conditions at the first end section, and introducing cell culture medium into the first end section to generate a positive pressure above ambient pressure onto the first surface of the carrier and the matrix material,
and withdrawing fluid from the second end section to generate a negative pressure below ambient pressure acting onto the second surface of the carrier oriented towards the second end section.

2. Implant according to claim 1, characterized in that a sealing is arranged at the perimeter of the carrier adjacent the circumferential inner surface of the bioreactor inner volume.

3. Implant according to claim 1, characterized in that the carrier is an integral portion of the porous matrix material.

4. Implant according to claim 1, characterized in that the carrier is in a fixed position within the bioreactor.

5. Implant according to claim 1, characterized in that the cells are present in admixture with an aqueous preparation of collagen.

6. Implant according to claim 1, characterized in that the second end section is in fluid connection with the first end section by a circulating line comprising a pumping means for generating the positive pressure at the first end section and the negative pressure at the second end section.

7. Implant according to claim 1, characterized in that a first mould covering the cross-section of the bioreactor is arranged between the carrier and the first end section, which first mould is pervious to fluid permeation and is arranged moveably within the bioreactor.

8. Implant according to claim 1, characterized in that the implant is selected from the group comprising bone tissue, bone tissue in combination with cartilage tissue, a meniscus, intervertebral discs, tendons and ligaments.

9. Implant according to claim 1, characterized in that the porous matrix material is selected from the group comprising acellularised spongiosa, acellularised veins, acellularised venules, acellularized tendons, acellularized ligaments, calcium-hydroxyl apatite, calcium-deficient hydroxyl apatite, β-tri-calcium phosphate and bioresorbable ceramics.

10. Implant according to claim 1, characterized in that the carrier is guided on a guide that generates a rotational movement in the carrier upon movement of the carrier along the axis between the first end section and the second end section.

11. Process for producing an implant, comprising the steps of providing a porous matrix material within a bioreactor under cell culture conditions,
providing a flow of cell culture medium comprising a first type of cells onto a porous matrix material and a positive pressure, acting onto a first side of the porous matrix material, characterized in that
negative pressure is applied to a second surface of the matrix material.

12. Process according to claim 11, characterized in that the first type of cells are present in an admixture with collagen.

13. Process according to claim 11, characterized in that a carrier is positioned adjacent the second side of the porous matrix material, the carrier being pervious to fluid permeation and extending over a section of the cross-section of the bioreactor, the carrier separating the bioreactor into a positive pressure section, in which the porous matrix material is arranged, and a negative pressure section.

14. Process according to claim 11, characterized in that the matrix material is cultivated under constant or alternating strain, which matrix material has a resilience, by fastening a first end of the matrix material adjacent a first end section of the bioreactor and fastening the opposed second end of the matrix material to the carrier.

15. Process according to claim 11, characterized in that the bioreactor is provided with at least one intermediate outlet opening arranged in the perimeter of the bioreactor between the first end section and the second end section, which intermediate outlet opening is connected by a fluid line with the circulating line connected to the second end section.

16. Process according to claim 12, characterized in that before adding the first type of cells in admixture with collagen, a second cell type is introduced into the bioreactor for colonizing the porous matrix material.

17. Process according to claim 16, characterized in that the first cell type is an aliquot of a puncture biopsy, and the second cell type is obtained by cultivating a second aliquot of the puncture biopsy for expansion of fibroblasts, fibrochondrocytes or chondrocytes.

18. Process according to claim 11, characterized in that the carrier is an integral portion of the porous matrix material.

19. Process according to claim 11, characterized in that the carrier is in a fixed position within the bioreactor.

20. Bioreactor for use in a process according to claim 11, the bioreactor comprising a reactor volume between a first end section and an opposed second end section, characterized by a carrier permeable to fluid permeation, which carrier extends across a portion of the cross-section of the bioreactor inner volume.

21. (canceled)

22. (canceled)
